(19) United States
(12) Patent Application Publication
(10) Pub. No.: US 2006/0047180 A1
(43) Pub. Date: Mar. 2, 2006

(54) Artificial sphincter

(76) Inventors: Anant V. Hegde, Newark, CA (US); George Yoseung Choi, Redwood City, CA (US); Wally S. Buch, Atherton, CA (US)

Correspondence Address: WILSON SONSINI GOODRICH & ROSATI, 650 PAGE MILL ROAD, PALO ALTO, CA, 94304-1050, US

(21) Appl. No.: 11/213,438

(22) Filed: Aug. 25, 2005

Related U.S. Application Data
(60) Provisional application No. 60/604,723, filed on Aug. 25, 2004.

(51) Int. Cl. A61F 2/02

(52) U.S. Cl. 600/30

(57) ABSTRACT

A biologically implantable artificial sphincter system and methods of using the same is disclosed. The artificial sphincter system disclosed herein comprises a support and an electroactive polymer element, both of which are adapted and configured to open and/or close a body cavity. The artificial sphincter systems are useful in the treatment of urinary incontinence, fecal incontinence, and reflux disorders. The implanted artificial sphincter can also provide a signal to the recipient to urinate or defecate.

### CROSS-REFERENCE

This application claims the benefit of U.S. Provisional Application No. 60/604,723, filed Aug. 25, 2004, which is incorporated herein by reference in its entirety.

### FIELD OF THE INVENTION

This invention relates to artificial sphincters, such as urinary, fecal and gastric sphincters, and methods of using the same.

### BACKGROUND

It is estimated that over 12 million Americans have urinary incontinence. Incontinence affects all ages, both sexes, and people of every social and economic level. It is also estimated that 15 to 30 percent of people over the age of 60 have incontinence. Women are twice as likely as men to have this condition. In addition, at least half of the 1.5 million Americans who reside in nursing homes are incontinent. The exact number of people with incontinence is not known, but the total number of people affected may be far greater than current estimates. Incontinence is a symptom that can be caused by a wide variety of conditions. Some of these causes, such as urinary tract or vaginal infections, medicine effects, or constipation, may be temporary. In addition, to urinary incontinence, fecal incontinence and reflux diseases are common disorders caused by malfunctioning sphincters.

Artificial sphincters that are in the market today have several components like pump, fluid reservoir, cuff, one-way valves and the tubing that connects the reservoir, pump and the cuff. It is not very comfortable for the patient to use these systems. Erosion, fluid loss, pressure loss, etc. compromise the effectiveness of the artificial sphincters over-time. Hence, there is a need to develop novel sphincters for use in disorders caused by the malfunction of natural sphincters in the body.

### SUMMARY OF THE INVENTION

The invention provides artificial sphincters and methods of use thereof. The artificial sphincter comprises a support and an electroactive polymer element for placement around a body cavity. The artificial sphincter can be used around several body cavities, including the urethra and various parts of the gastro-intestinal tract. The sphincter system allows for opening and/or closing of the body cavity around which it is placed, this opening and closing being controlled by the activation of the electroactive polymer element with electrical signals. The artificial sphincter system can also include a sensor to sense the state of the body cavity it surrounds to provide signals for activation or inactivation of the electroactive polymer element.

A first aspect of the invention is an artificial sphincter comprising an electroactive polymer element and a support, both of which being configured to allow the constriction of a body cavity between the electroactive polymer element and the support. The artificial sphincter is useful for constriction of various body cavities, including the urethra to treat urinary incontinence; the esophagus to treat reflux disease, and the rectum to treat fecal incontinence. The sphincter further comprises electrical terminals contacting the electroactive polymer element for modulating the shape of the electroactive polymer element. The support can be rigid or semi-rigid such as to provide a certain amount of stiffness for constricting the body cavity between the electroactive polymer element and the support.

Preferably, the artificial sphincter comprises a support in the form of an enclosure with a lumen, for placement around a body cavity, and an electroactive polymer element. The enclosure can also include a soft elastomeric layer around the lumen.

In some embodiments, the artificial sphincter includes a control unit for electrically controlling the electroactive polymer element to open or close the body cavity. The action of the artificial sphincter of the present invention can be controlled using a variety of control units, for example, (a) power source and a simple switch or (b) power source and a logic/control device such as a computer. The artificial sphincters of the present invention can also comprise a sensing system (such as a system comprising strain gauges) for sensing the degree of deformation of the electroactive polymer element.

In one embodiment of the invention, the artificial sphincter has a rigid enclosure with a through-lumen with soft elastomeric layer, an electroactive polymer element, power switch, leads and a power source. Optionally there is a deformable element, such as a compression spring, inside the enclosure. The deformable element pushes the elastomeric layer outward to keep the through-lumen closed. One end of the electroactive polymer element is in between the deformable element and the elastomeric layer, and the other end is secured inside the enclosure to a terminal block. The terminal block is connected to the power source through lead wires via a switch. The sphincter is placed around a body cavity, such as the urethra, and the soft elastomeric layer comes in contact with the wall of the body cavity, such as the urethra wall. At rest, the polymer element is not charged and the body cavity, such as the urethra, remains closed. When the power is delivered by depressing the switch, the polymer element deflects inward and activates the deformable element, thus allowing the body cavity, such as the urethra, to open in order to empty the bladder. When the power is stopped, the polymer will lose its charge and lose the strength to keep the deformable element activated. The deformable element returns to its normal state and again closes the body cavity.

In one embodiment of the invention, the power source and the switch are implanted in the patient's body just beneath the outer skin. This embodiment may also include a battery recharging mechanism implanted in the patient's body. In another embodiment of the invention, the power source is outside the patient's body and the power is transmitted transcutaneously through the induction coil that is implanted in the patient's body. In another embodiment of the present invention, the actuator used is a superelastic shape memory alloy like Nitinol.

In one embodiment, the invention comprises of a biologically implantable artificial sphincter comprising an electroactive polymer element; a support; and a conduit having a first side and a second side; wherein electroactive polymer element is on the first side of the conduit. Preferably, the support is on the second side of the conduit and the first side of the conduit is substantially opposite to the second side of the conduit. The conduit can be circumscribed by an elastomeric sheath.

Preferably, the electroactive polymer element comprises an ion-exchange polymer metal composite. The electroactive polymer element can be in the shape of a panel, which is substantially flat or in the shape of a spring. The sphincter can further comprise a deformable element, such as a compression spring, which is in mechanical communication with the electroactive polymer element. Preferably, the sphincter also includes a power supply in electrical communication with the electroactive polymer element and a switch.

A second aspect of the invention, is a method of opening and/or closing a body cavity using an artificial sphincter described herein. For example, one embodiment is a method of treating urinary incontinence using an artificial sphincter comprising implanting the artificial sphincter around the urethra; closing the urethra with mechanical spring force in the artificial sphincter; and opening the urethra by transmitting an electrical signal to the artificial sphincter; wherein opening the urethra comprises the electrical signal actuating an electroactive polymer element in the artificial sphincter. Preferably, the closing of the urethra is via constricting the urethra between the electroactive polymer element and a support. The urethra can be closed by not transmitting electricity to the electroactive polymer element and opened by using a mechanical spring force to pull the electroactive polymer element away from the urethra. The artificial sphincters described herein can also be used for the treatment of fecal incontinence and reflux diseases.

The artificial sphincter cuffs of the present invention may be adapted for placement around a number of body lumens, including the urethra, the anal canal, and the lower esophagus.

One embodiment of the invention is an artificial sphincter comprising an electroactive polymer element and a support, said electroactive polymer element and support being configured to constrict a body cavity disposed therebetween. The support of the sphincter can be an encapsulating device with one passage for a body organ. This passage can be substantially circumscribed by a sheath. The electroactive polymer element can be a substantially flat surface or a spring. This embodiment can further a spring in mechanical communication with the electroactive polymer element. The sphincter also includes a power supply in electrical communication with the electroactive polymer element.

Another embodiment of the invention is an implantable control device comprising an electroactive polymer actuator, an enclosure, and a power management device; wherein the enclosure is configured to encompass a body cavity, the electroactive polymer actuator and the enclosure are configured to constrict the body cavity, and the power management device is adapted to connect to the electroactive polymer actuator.

The devices disclosed herein can be coated with materials to prevent or promote tissue growth. Also, the devices can include an inductive coupling mechanism adapted to connect the electroactive polymer to a power source. The body cavities regulated by the sphincters disclosed herein include urethra, lower esophagus, lower gastro-intestinal tract, or rectum.

Another embodiment of the invention is a method of controlling passage of contents across a body cavity comprising implanting a control device around a body cavity, the device comprising an electroactive polymer actuator, an enclosure, and a power management device; controlling a flow of contents in the body cavity, this control being performed by constricting and unconstricting of the body cavity between the electroactive polymer actuator and the enclosure. In this method control of flow of contents in the body cavity can be in response to transcutaneous feedback from the body cavity, said feedback being related to the contents in the body cavity. The control device described herein can be controlled with an inductive coupling mechanism. The inductive coupling mechanism can be transcutaneous.

The devices described herein are suitable for the treatment of several disorders such as disorders of the urethra, lower esophagus, lower gastro-intestinal tract, or rectum. One embodiment is a method of treating a disease using an artificial sphincter comprising implanting an artificial sphincter around a body cavity, the artificial sphincter comprising an electroactive polymer element and a support; closing the body cavity with the artificial sphincter by applying a mechanical force on the body cavity between the support and the electroactive polymer element; and opening the body cavity by transmitting an electrical signal to the electroactive polymer element. This method can be used in the treatment of urinary incontinence, fecal incontinence, or reflux diseases.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A and 1B illustrate a male and female urinary system.

FIGS. 2A and 2B illustrate an embodiment of the artificial sphincter system in use in a female and a male urinary system.

FIGS. 3A-3D illustrate embodiments of the artificial sphincter system.

FIG. 4 illustrates an embodiment of the artificial sphincter system.

FIG. 5 illustrates a cross-sectional view of the upper gastro-intestinal tract.

FIG. 6 illustrates an embodiment of the artificial sphincter system in use in upper gastro-intestinal tract.

FIG. 7 illustrates a cross-sectional view of the lower gastro-intestinal tract.

FIG. 8 illustrates a cross-sectional view of an embodiment of the artificial sphincter in use in a lower gastro-intestinal tract.

FIG. 9 illustrates an embodiment of an inductive coupling system associated with the artificial sphincter system.

FIG. 10 illustrates an embodiment of an inductive coupling system associated with the artificial sphincter system.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Artificial Sphincter System

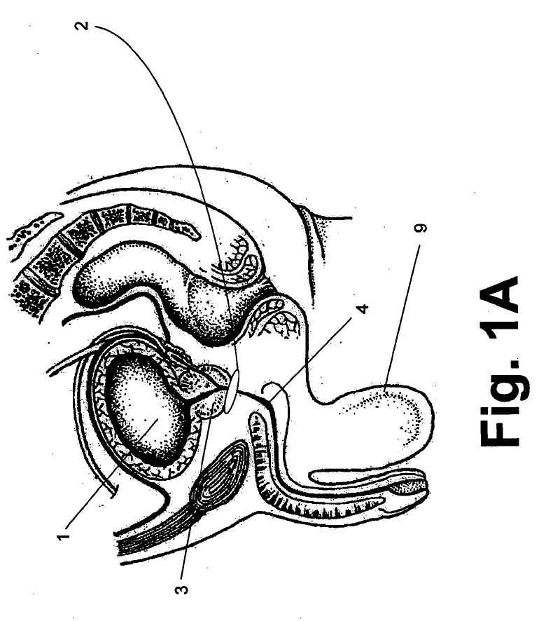

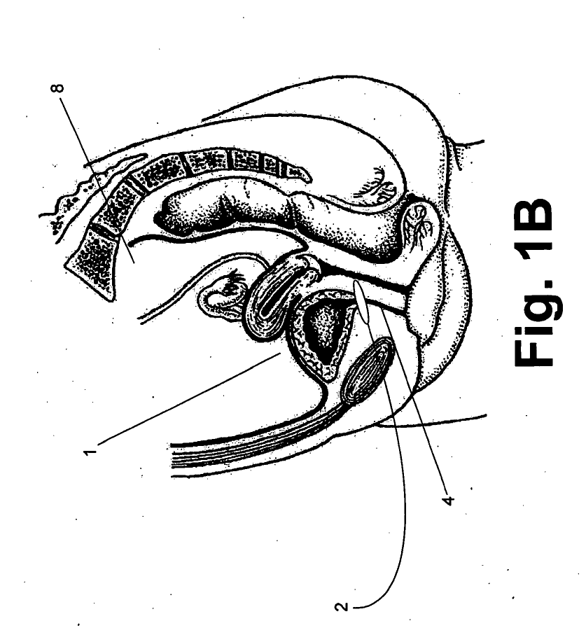

FIGS. 1A and 1B depict the male and female urinary system. Some of the components of a male urinary system, as depicted in FIG. 1A, are the urinary bladder 1, prostate gland 3, urinary sphincter muscle 2, urethra 4, and scrotum 9. The components of a female urinary system, as depicted in FIG. 1B, are the urinary bladder 1, uterus 8, urinary sphincter muscle 2, and urethra 4.

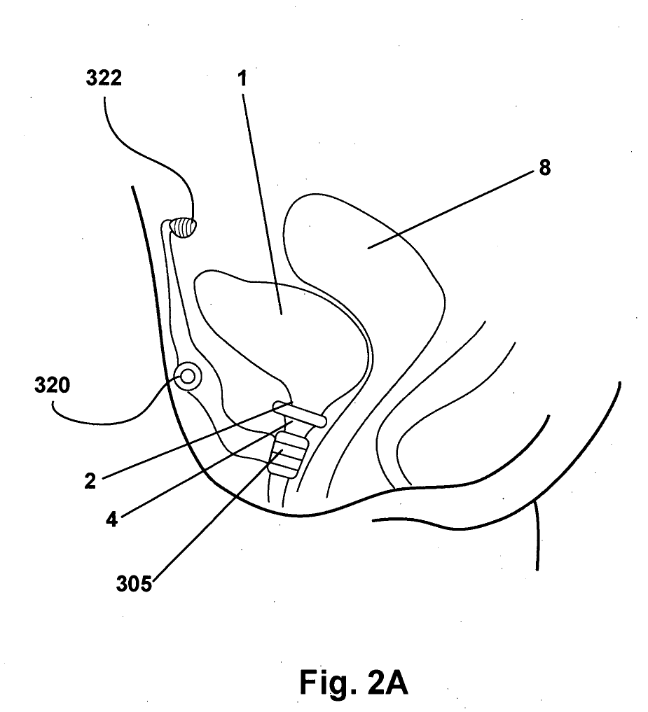

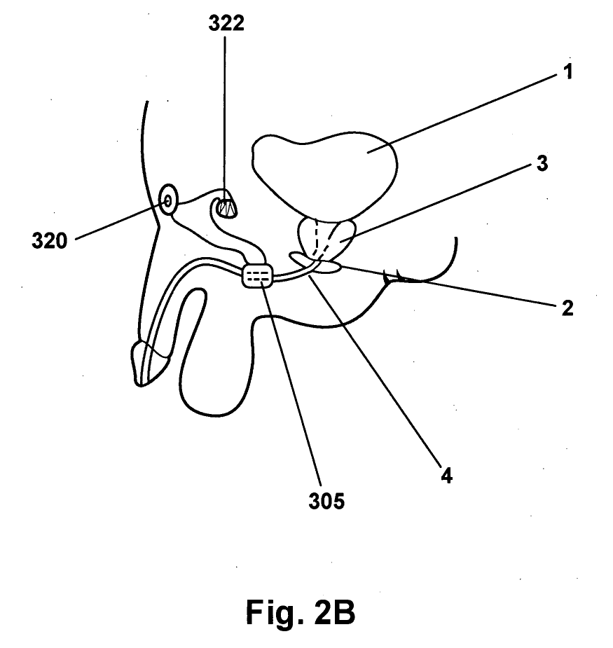

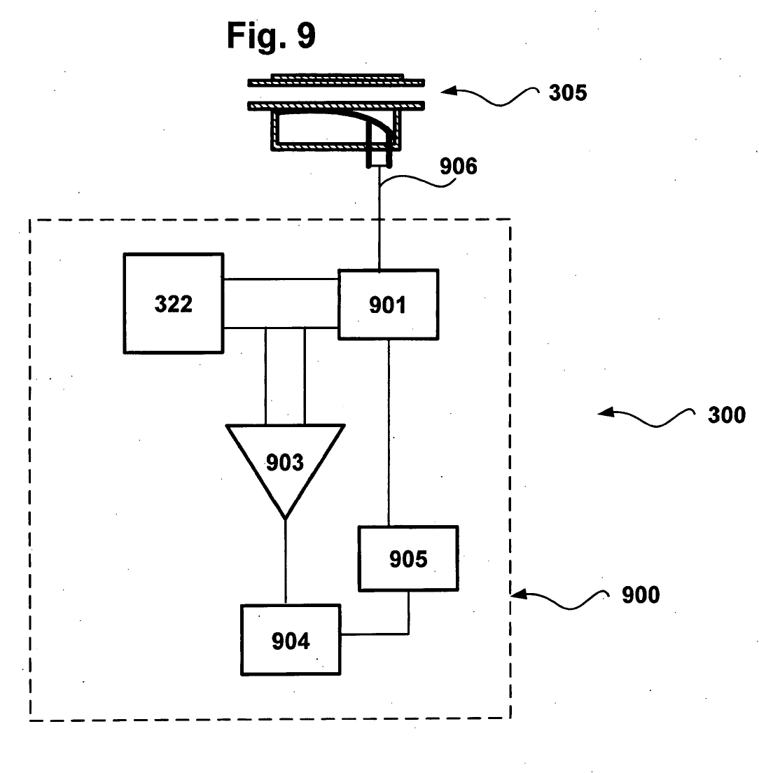

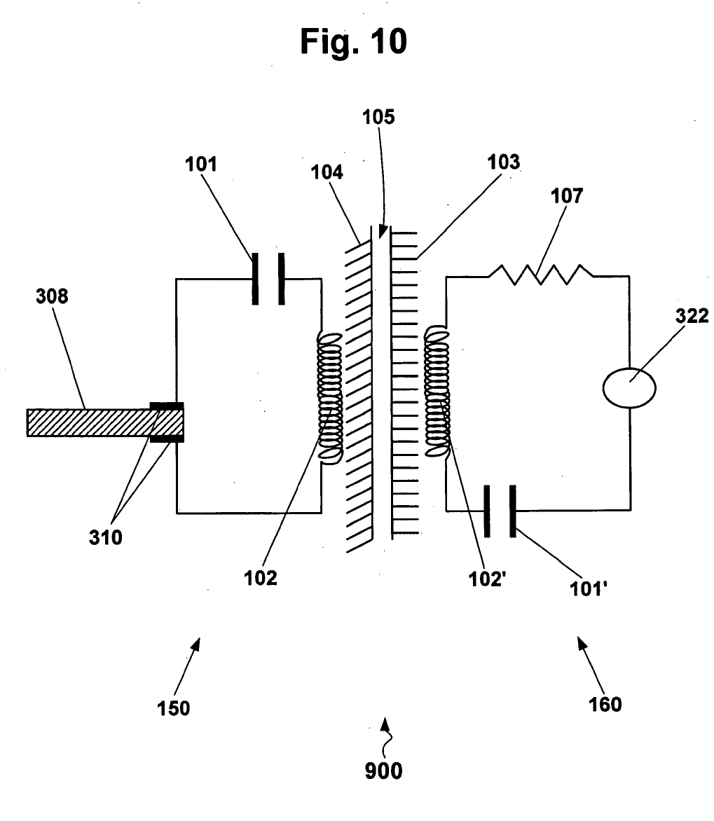

FIG. 2A illustrates an embodiment of an artificial sphincter system 300 implanted in a female subject. The artificial sphincter system 300 discussed herein comprises of the artificial sphincter 305 or 400 and an inductive coupling system 900 such as depicted in FIGS. 9 and 10. The artificial sphincter system in the female subject is similar to the artificial sphincter system shown for the male subject in FIG. 2B. In the illustrated embodiment, in FIGS. 2A and 2B, the artificial sphincter 305 is controlled with a switch 320 and a power source 322. The switch and power source may be located inside or outside the body.

FIG. 2B illustrates an embodiment of an artificial sphincter system 300 implanted in a male subject. The subject has a bladder 1, a sphincter muscle 2, a prostate gland 3 and a urethra 4. As depicted, the artificial sphincter system 300 has an artificial sphincter 305, a switch 320, and a power source 322. The switch 320 and/or power source 322 can be connected to the artificial sphincter 305.

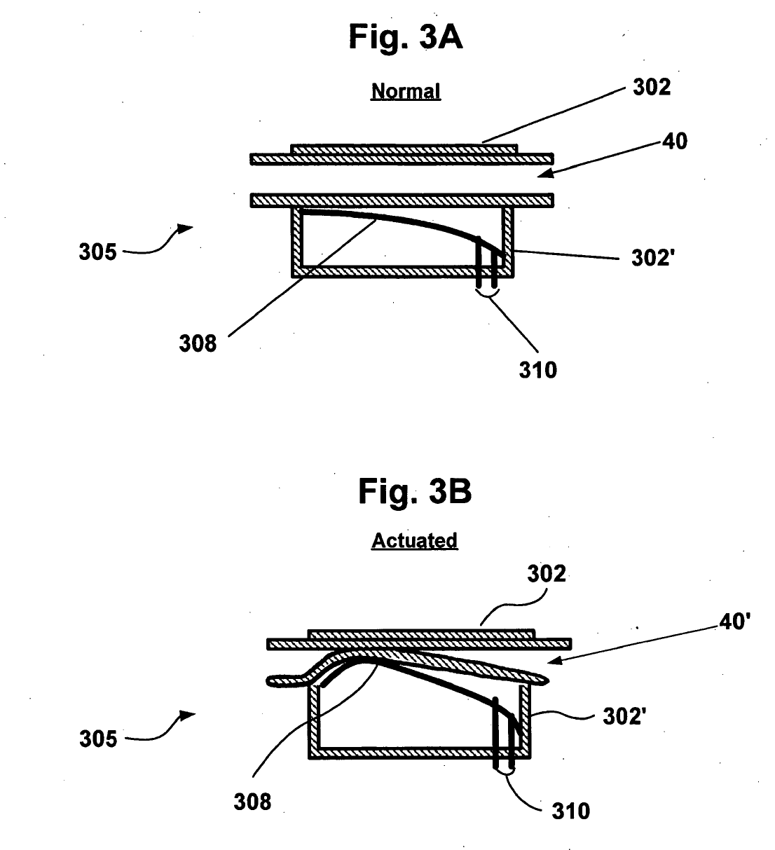

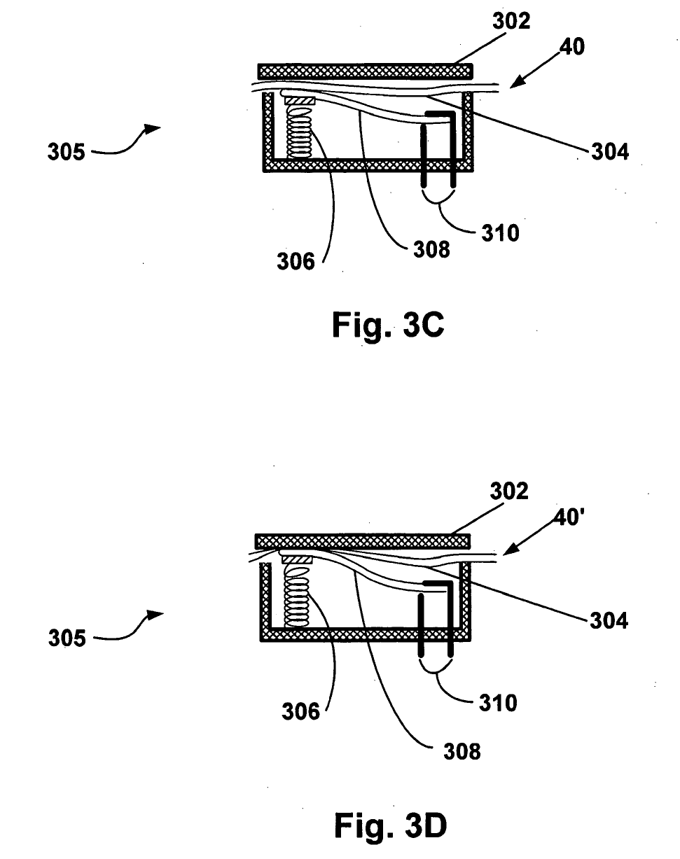

FIGS. 3A-3D depict two embodiments of an artificial sphincter system. FIGS. 3A and 3B depict an embodiment of an artificial sphincter 305 with a support 302 and an enclosure 302′ containing the electroactive polymer element 308. The electroactive polymer element 308 has electric contacts 310. The support 302, enclosure 302′, and electroactive polymer element 308 are configured and adapted to constrict and unconstrict a body cavity 40. In the normal state depicted in FIG. 3A, the electroactive polymer element 308 is not activated and leaves the body cavity 40 open, i.e., unconstricted. In the actuated state in FIG. 3B, the electroactive polymer element 308 is activated by the electrical contacts 310 and closes the body cavity 40′, i.e., constricted. Examples of body cavities around which the artificial sphincter system 300 can be used include the urethra and gastro-intestinal cavities, such as the esophagus, the large intestine and the rectum. In other embodiments, the body cavity 40 can be constricted by the electroactive polymer element 308 when it is in its normal, non-activated state and the body cavity can be opened when the electroactive polymer element 308 is actuated by activation by the electrical contacts 310. The support 302 and the enclosure 302′ can be configured as a single piece or as multiple pieces.

FIGS. 3C and 3D illustrate a cross-sectional view of the artificial sphincter 305 that is configured to close the body cavity 40 when it is at rest. Artificial sphincter 305 at rest is depicted in FIG. 3D. The artificial sphincter 305 has a support 302 and an electroactive polymer element 308. The support 302 is an enclosure such as an outer shell with a lumen or a clam shell with a lumen or just a support. The support is rigid or is semi-rigid such as to provide a certain amount of stiffness for constricting the body cavity 40 between the electroactive polymer and the encapsulating device. As shown in FIGS. 3C and 3D, the artificial sphincter 305 includes a soft elastomeric layer 304, a deformable element 306, such as the compression spring shown in FIGS. 3C and 3D, an actuator such as an electroactive polymer (EAP) element 308, and a power supply terminal 310. FIG. 3C illustrates a cross-sectional view of the artificial sphincter 305 that is configured to open up the body cavity 40 when activated.

In the embodiment depicted in FIGS. 3C and 3D, the body cavity 40 is closed when there is no electrical current applied to the EAP element 308. In this state, as depicted in FIG. 3D, the deformable element applies mechanical force on the EAP element 308, and the body cavity 40′ is constricted closed between the EAP element 308 and the support 302. When electrical current is applied on the EAP element 308, as depicted in FIG. 3C, the EAP element 308 is deflected away from the body cavity 40 and the body cavity 40 is opened. In an alternative embodiment of the artificial sphincter system 305, the body cavity 40′ is closed when electrical current is applied to the EAP element 308, and the EAP element 308 constricts the body cavity 40 closed between itself and the support 302. The body cavity, in this embodiment, is opened when the electrical current is not applied to the EAP element 308, and the EAP element moves away from the body cavity 40 to unconstrict it and thus open it up.

The electroactive polymer element 308 has two power supply terminals 310. The power supply terminals 310 (e.g., an anode and a cathode) connect to the surface of the EAP element 308. When the EAP element 308 is activated, the EAP element 308 deforms. In the embodiments shown in FIGS. 3C and 3D, the deformation of the EAP element 308 activates the deformable element 306. The activation of the deformable element 306, such as a compression spring, removes the constriction pressure from the body cavity 40′ and allows the body cavity 40 to open. The open body cavity 40 allows contents of the body cavity, such as urine or feces, to pass through the body cavity 40. When the body cavity 40 is empty, removing the charge to the EAP element 308 allows the deformable element 306 to relax. The relaxed deformable element 306 closes (e.g., constricts) the body cavity 40, for example, against a support such as the inside of the support 302. Support 302 may be covered with an elastomer or a biocompatible and/or non-abrasive coating.

In some embodiments, the EAP element 308 is an ion-exchange polymer metal composite (IPMC). The element 308 is encapsulated in the outer shell 302. The outer shell 302 has one or more openings. A conduit is defined between the openings. The body cavity 40 passes through the openings and the conduit, and the outer shell 302. The artificial sphincter 305 is configured so the EAP element 308 does not directly contact the body cavity 40′. The elastomeric layer 304 separates the EAP element 308 from the body cavity 40. The EAP element 308 is single layered or multi-layered.

The elastomeric layer 304 can be made of silicone, latex, polychloroprene (e.g., neoprene), fully vulcanized thermoplastic rubbers (TPRs) such as polyolefin-based or styrene-based rubbers (e.g., Alcryn® from Dupont, Kryton® from Shell, Santoprene® from Monsanto), thermoplastic elastomers (TPEs) such as polyester TPEs or nylon TPEs (e.g., Hytel® from Dupont, Lomod® from GE, Pebax® from Elf AtoChem) or any other thermoplastic or thermosetting plastic polymer.

In some embodiments, the artificial sphincter is controlled with a transcutaneous energy transmission system (TETS) and/or a processor. The TETS transmitter coil (not shown) would preferably be located outside the body. The processor is configured to control the artificial sphincter and also sense and/or process other relevant information necessary for control of the body cavity, such as the urethra.

In some embodiments, a power supply 322 (e.g., a battery) and an ON/OFF switch 320 are implanted in the subject. The power supply 322 and the switch 320 can be anywhere in the subject that is convenient to the subject. Wires connect the power supply 322, the switch 320 and the artificial sphincter 305. In other embodiments, the power supply 322 is outside the body of the subject. In such embodiments power can be transmitted to the artificial sphincter 305 using a transcutaneous energy transfer system (TETS), for example a system that inductively transmits energy (i.e., similar to methods for delivering power to artificial hearts).

FIG. 9 depicts an inductive coupling system that is suitable for controlling the artificial sphincter 305 which includes a connecting element 906 (which connects the electrical contacts 310 to the rest of the electrical system), a connector 901, a energy source 322, a sensor 903, a timer 904, and a controller 905. The connector 901, energy source 322, sensor 903, a timer 904, and controller 905 are located in a housing disposed in a region outside or inside the body.

FIG. 10 depicts one embodiment of an electrical system 900 associated with a EAP element 308. The inductive coupling system 900 has an implanted portion 150 and a non-implanted portion 160. The implanted portion 150 is a closed circuit with the first inductor 102 in series with a first capacitor 101 and the EAP element 308. The EAP element 308 is attached to the closed circuit of the implanted portion 150 by electrical contacts 310. The implanted portion is a closed circuit and can have a resistor (not shown). The non-implanted portion 160 has a second inductor 102′ that is in series with a resistor 107, the power supply 322, and a second capacitor 101′. The capacitors, resistors, and, in part, the inductors are representative of the electrical characteristics of the wire of the circuit and are not necessarily representative of specific elements. The implanted portion 150 is within tissue and has a tissue surface 104 nearby. The non-implanted portion is in insulation material 103. An air interface 105 is between the tissue surface 104 and the insulation material 103.

The power supply 322 can be a power cell, a battery, a capacitor, a substantially infinite bus, a portable generator, or combinations thereof. The power supply typically has a power output of from about 1 mA to about 5 A. The connecting element 906 is a wire lead, an inductive energy transfer system, a conductive energy transfer system, a chemical transfer system, an acoustic or otherwise vibratory energy transfer system, a nerve or nerve pathway, other biological tissue, or combinations thereof. The connecting element is made from one or more conductive materials, such as copper. The connecting element is completely or partially insulated and/or protected by an insulator, for example polytetrafluoroethylene (PTFE). The insulator is typically biocompatible. The power supply 322 is in electrical communication with the EAP element 308 through a connecting element. The connecting element is attached to the electrical contacts 310.

In other embodiments, the EAP element 308 can be wrapped around the body cavity 40 and use the deformable element 306, such as compression springs, in series. Using the EAP element 308 in series with the compression spring 306 can achieve the same function as the configuration described supra and in FIGS. 3A-3D.

The EAP element 308 and the surface of any other elements described herein can be coated with materials and/or agents to promote tissue growth around the coated surfaces. The EAP element 308 and the surface of any other elements described herein can be coated with materials and/or agents to eliminate and/or prevent tissue growth around the coated surfaces.

The actuator can be a superelastic Nitinol material instead of an IPMC. The actuator can be a leaf spring. The actuator can be in the artificial sphincter without the deformable element 306 attached to the actuator.

The artificial sphincter can be controlled with a sensor and a controller to open and close the body cavity, such as the urethra. The controller can be a programmable device to open and close the body cavity via the electroactive element of the artificial sphincter. The sensor can be a pressure sensing device that can sense the pressure in the body cavity, such as urinary bladder or gastro-intestinal tract, and send a signal to the controller.

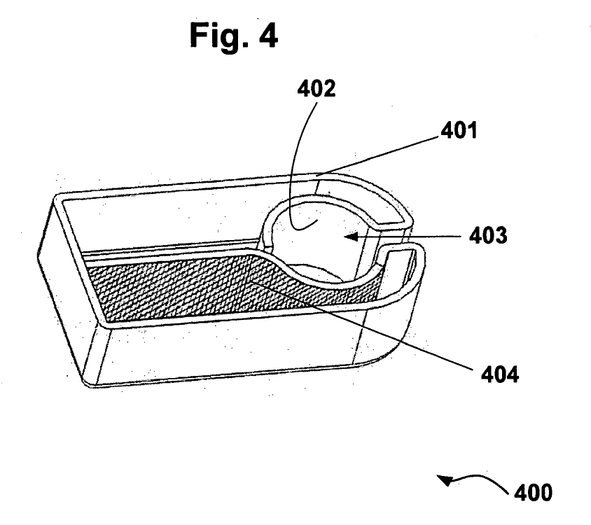

Another embodiment of the artificial sphincter 400 is shown in FIG. 4. This embodiment comprises a clam-shell shaped enclosure 401, support 402, a lumen for the body cavity 403, and an electroactive polymer element 404. The support 402 can be rigid or semi rigid, the rigidity level being such that it allows for constriction of the body cavity by the support 402 and the electroactive polymer element 404. FIG. 4 depicts the artificial sphincter 400 with a wall removed so as to view the interior of the device. Preferably, the device is a single piece along with the top (which is not shown). The artificial sphincter 400, typically, includes a hinging mechanism to allow its placement around a body cavity. The two ends of the clam-shell shaped enclosure 401 can come around to meet to provide a snug fit around the body cavity, which would be present in the lumen 403. The clam-shell shaped enclosure 401 and support 402 can be made of different materials. Further, the clam-shell shaped enclosure 401 and support 402 can be one continuous piece or can be separate pieces. The artificial sphincter 400 controls the constriction of the body cavity present in lumen 403 by the movement of the electroactive polymer element 404 towards the support 402 such that mechanical force is applied on the body cavity present in lumen 403.

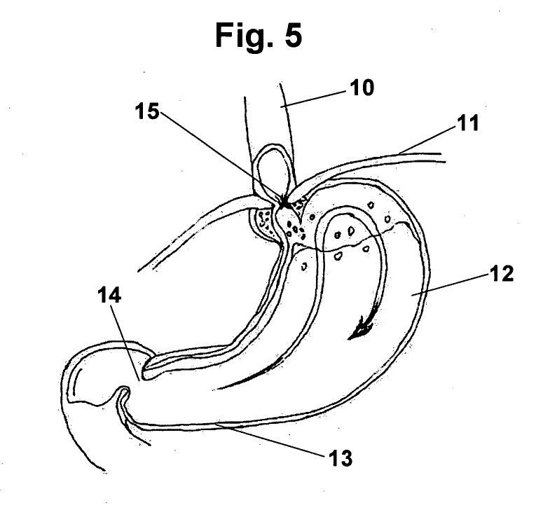

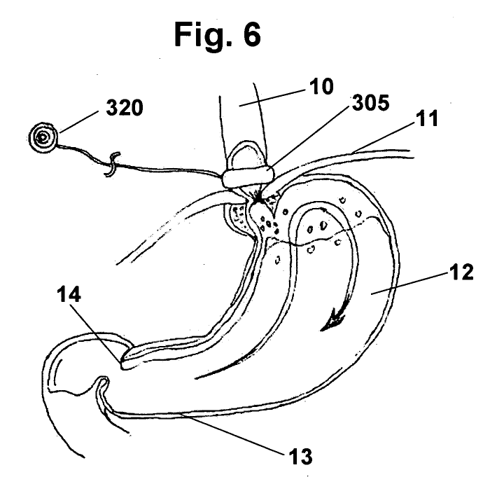

FIG. 5 depicts the upper gastro-intestinal tract with the esophagus 10, lower esophagus sphincter 15, diaphragm 11, stomach 13, liquid contents 12, and pylorus 14. FIG. 6 depicts the use of an artificial sphincter system 300 or 400 in the esophagus for the treatment of reflux disorders with the artificial sphincter 305 and switch 320.

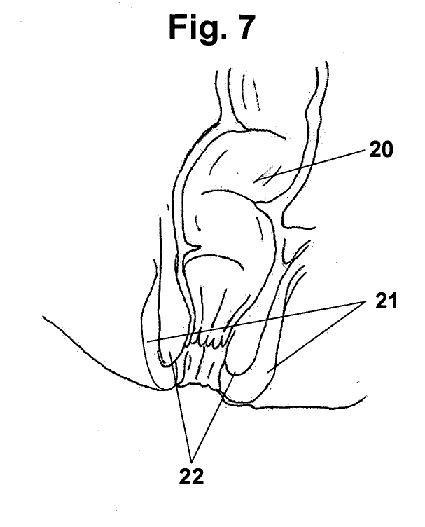

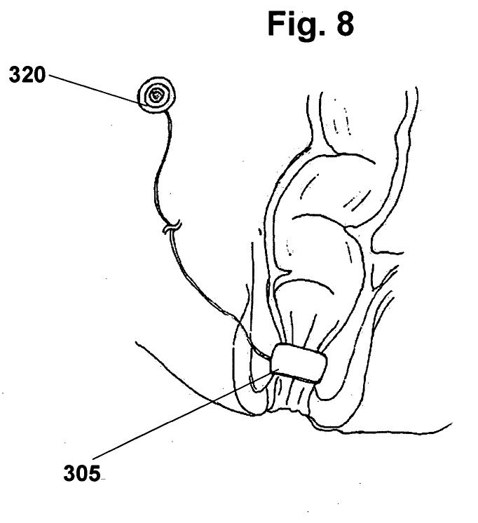

FIG. 7 depicts the lower gastro-intestinal tract with the rectum 20, exterior sphincter 21, and interior sphincter 22. FIG. 8 depicts the use of an artificial sphincter system 300 or 400 in the rectum for the treatment of fecal incontinence with the artificial sphincter 305 and switch 320.

The devices described herein maybe implanted with or without sutures or other bonding material such as surgical glue. The devices in some embodiments have external fibers or surface pores or coatings, such as protein based coatings like poly-L-lysine and poly-D-lysine, to promote tissue in-growth and help affix the device to adjacent tissue. In other embodiments, the devices are coated with material to prevent tissue growth around the implanted device, such as hyaluronic acid.

U.S. Pat. No. 6,749,556 to Banik is hereby incorporated by reference in its entirety.

Methods of Making EAP Element

In some embodiments, the EAP element 308 is an IPMC strip which is made from a base material of an ionomer sheet, film or membrane. The ionomer sheet is formed using ionomer dispersion.

IPMC is made from the base ionomer of, for example, polyethylene, polystryrene, polytetrafluoroethylene, polyvinylidene fluoride (PVDF) (e.g., KYNAR® and KYNAR Flex®, from ATOFINA, Paris, France, and SOLEF®, from Solvay Solexis S.A., Brussels, Belgium), hydrophilic-PVDF (h-PVDF), polyfluorosulfonic acid based membranes like NAFION® (from E.I. Du Point de Nemours and Company, Wilmington, Del.), polyaniline, polyacrylonitrile, cellulose, cellulose acetates, regenerated cellulose, polysulfone, polyurethane, and combinations thereof. The conductive material that is deposited on the ionomer can be gold, platinum, silver, palladium, copper, graphite, conductive carbon, or combinations thereof. Conductive material is deposited on the ionomer either by electrolysis process, vapor deposition, sputtering, electroplating, or combination of processes.

The IPMC is cut into the desired implant shape for the EAP element 308. The electrical contact 310 (e.g., anode and cathode wires for EAP element) is connected to the IPMC surfaces by, for example, soldering, welding, brazing, potting using conductive adhesives, or combinations thereof. The EAP element 308 is configured, if necessary, into specific curved shapes using mold and heat setting processes.

In some embodiments, the EAP element 308 is insulated with electrical insulation coatings. Also, the EAP element 308 can be insulated with coatings that promote cell growth and minimize fibrosis, stop cell growth, or kill nearby cells. The insulation can be a biocompatible material. The EAP element 308 is coated with polymers such as polypropylene, poly-L-lysine, poly-D-lysine, polyethylene glycol, povinyl alcohol, polyvinyl acetate, polymethyl methacrylate, or combinations thereof. The EAP element 308 can also be coated with hyaluronic acid. The coating is applied to the device by standard coating techniques like spraying, electrostatic spraying, brushing, vapor deposition, dipping, etc.

In one example, a perfluorosulfonate ionomer, PVDF or h-PVDF sheet is prepared for manufacturing the EAP element 308. The sheet is roughened on both sides using, for example, about 320 grit sand paper and then about 600 grit sand paper. The sheet is then rinsed with deionized water. The sheet is then submerged in isopropyl alcohol (IPA), and subjected to an ultrasonic bath for about 10 minutes. The sheet is rinsed with deionized water. The sheet is then boiled for about 30 minutes in hydrochloric acid (HCL). The sheet is rinsed and then boiled in deionized water for about 30 minutes. The sheet is then subject to ion-exchange (i.e., absorption). The sheet is submerged into, or otherwise exposed to, a metal salt solution at room temperature for more than about three hours. Examples of the metal salt solution are tetraammineplatinum chloride solution, silver chloride solution, hydrogen tetrachloroaurate, tetraamminepalladium chloride monohydrate or other plantinum, gold, silver, carbon, copper, or palladium salts in solution. The metal salt solution typically has a concentration of greater than or equal to about 200 mg/100 ml water. 5% ammonium hydroxide solution is added at a ratio of 2.5 ml/100 ml to the tetraamineplatinum chloride solution to neutralize the solution. The sheet is then rinsed with deionized water. A primary plating is then applied to the sheet. The sheet is submerged in water at about 40° C. A 5% solution by weight of sodium borohydride and deionized water is added to the water submerging the sheet at 2 ml/180 ml of water. The solution is stirred for 30 minutes at 40° C. The sodium borohydride solution is then added to the water at 2 ml/180 ml of water and the solution is stirred for 30 minutes at 40° C. This sodium borohydride adding and solution stirring is performed six times total. The water temperature is then gradually raised to 60° C. 20 ml of the sodium borohydride solution is then added to the water. The solution is stirred for about 90 minutes. The sheet is then rinsed with deionized water, submerged into 0.1 N HCI for an hour, and then rinsed with deionized water.

In some embodiments, the sheet receives a second plating. The sheet is submerged or otherwise exposed to a tetraamineplatinum chloride solution at a concentration of about 50 mg/100 ml deionized water. 5% ammonium hydroxide solution is added at a rate of 2 ml/100 ml of tetrammineplatinum chloride solution. 5% by volume solution of hydroxylamine hydrochloride in deionized water is added to the tetraammineplantium chloride solution at a ratio of 0.1 of the volume of the tetraammineplatinum chloride solution. 20% by volume solution of hydrazine monohydrate in deionized water is added to the tetraammineplatinum chloride solution at a ratio of 0.05 of the volume of the tetraammineplantinum chloride solution. The temperature is then set to about 40° C. and the solution is stirred.

A 5% solution of hydroxylamine hydrochloride is then added at a ratio of 2.5 m/100 ml of tetraamnineplatinum chloride solution. A 20% solution of hydrazine monohydrate solution is then added at a ratio of 1.25 ml/100 ml tetraammineplatinum chloride solution. The solution is stirred for 30 minutes and the temperature set to 60° C. The above steps in this paragraph can be repeated three additional times. The sheet is then rinsed with deionized water, boiled in HCl for 10 minutes, rinsed with deionized water and dried.

In some embodiments, the polymer base is dissolved in solvents, for example dimethyl acetamide, acetone, methylethyle ketone, toluene, dimethyl carbonate, diethyl carbonate, and combinations thereof. The solvent is then allowed to dry, producing a thin film. While the solution is wet, a low friction, (e.g., glass, Teflon) plate is dipped into the solution and removed. The coating on the plate dries, creating a think film. The plate is repeatedly dipped into the solution to increase the thickness of the film.

Polyvinyl alcohol, polyvinyl pyrrolidone, polyinyl acetate or combinations thereof can be added to a PVDF solution before drying, thus contributing hydrophilic properties to PVDF and can improve ion migration through the polymer film during manufacture. Dye or other color pigments can be added to the polymer solution.

Treatment of Diseases

The artificial sphincter systems disclosed herein are suitable for treatment of several diseases. These diseases include diseases caused by the malfunctioning of a body cavity and the resultant effects on the contents of the body cavity. Such diseases are typically caused due to the malfunctioning of sphincters and or valves that control these body cavities and/or due to the malfunctioning of peristaltic activity of the body cavity. Typically, these body cavities are tubular organs, such as the urethra, the gastro-intestinal tract, and blood vessels. The artificial sphincters described herein are placed around a body cavity, such as a urethra, gastro-intestinal tract, and blood vessels. The diseases that can be treated include urinary incontinence, fecal incontinence, and reflux disorders. These sphincters are used by themselves or are used in combination with conventional therapies, including drugs, dietary modifications, and/or surgery. The sphincters are also suitable for prophylactic uses.

Urinary Incontinence

Urine is waste and water removed from the blood by the kidneys. Urine flows from the kidneys downward through a pair of tubes (the ureters) to the bladder. The bladder is a balloon-like container that stores urine. Urine leaves the body through another tube (the urethra) at the bottom of the bladder.

Urination is controlled by muscles, called sphincters, located at the base of the bladder and in the wall of the urethra. These normally stop the flow of urine. Usually, the sphincters close off the neck of the bladder and the urethra-like a tie around the bottom of a balloon—so that urine does not leak. When sphincters relax, they open the passage for urine. At the same time, the muscle of the bladder wall contracts (squeezes) and forces the urine out of the bladder. When urination is finished, the sphincters contract, and the bladder itself stops squeezing and relaxes.

Urinary incontinence is the medical term used to describe the condition whereby patient cannot control the flow of urine from the body. It usually happens because the sphincter is damaged. A damaged sphincter can not squeeze and close off the urethra. This means urine can leak or flow freely from the bladder. Many things can prevent the sphincter and bladder from doing their jobs. Most frequently, incontinence occurs in men when the sphincter and its nerves are affected by total or partial removal of the prostate to treat prostate cancer or other conditions. Sometimes an oversensitive or small bladder can put too much pressure on an otherwise healthy sphincter. Some other conditions include: urinary tract or vaginal infections, effects of medicine, constipation, weakness of certain muscles, blocked urethra due to an enlarged prostate, diseases and disorders involving nerves and/or muscles, and some types of surgery. Other causes can be longer-lasting, even permanent. These include such conditions as an overactive bladder muscle, weakness of the muscles holding the bladder in place, weakness of the sphincter muscles surrounding the urethra, birth defects, spinal cord injuries, surgery, or diseases involving the nerves and/or muscles (multiple sclerosis, muscular dystrophy, polio, and stroke). In some cases, more than one factor causes incontinence in a single individual.

Many types of treatment are available for incontinence depending on the type of incontinence one has. If the incontinence is due to the weakness of the sphincter muscle, artificial sphincter can be implanted to aid or replace the sphincter muscle.

The artificial sphincter disclosed herein can be used to replace the patient's natural sphincters and when patient feels the need to pass urine; patient has to activate the sphincter by simply applying the power to the sphincter actuator transcutaneously. The sphincter can be used alone or in combination with other conventional treatments for urinary incontinence.

Fecal Incontinence

Fecal incontinence is the inability to control bowels. When one feels the urge to have a bowel movement, may not be able to hold it until one can get to a toilet or stool may leak from the rectum unexpectedly.

Fecal incontinence can have several causes including, but not limited to, constipation, damage to the anal sphincter muscles, damage to the nerves of the anal sphincter muscles or the rectum, loss of storage capacity in the rectum, diarrhea, and pelvic floor dysfunction. Fecal incontinence can be caused by injury to one or both of the ring-like muscles at the end of the rectum called the anal internal and/or external sphincters. The sphincters keep stool inside. When damaged, the muscles aren't strong enough to do their job, and stool can leak out. In women, the damage often happens when giving birth. The risk of injury is greatest if the doctor uses forceps to help deliver the baby or does an episiotomy, which is a cut in the vaginal area to prevent it from tearing during birth. Hemorrhoid surgery can damage the sphincters as well.

Treatment depends on the cause and severity of fecal incontinence; it may include dietary changes, medication, bowel training, or surgery. More than one treatment may be necessary for successful control since continence is a complicated chain of events. Food affects the consistency of stool and how quickly it passes through the digestive system. If patient's stool is hard to control because it is watery, may find that eating high fiber foods adds bulk and makes stool easier to control. But people with well-formed stools may find that high fiber food act as a laxative and contribute to the problem. Other food that may make the problem worse are drinks containing caffeine, like coffee, tea, and chocolate, which relax the internal anal sphincter muscle. If diarrhea is causing the incontinence, medication may help. Sometimes doctors recommend using bulk laxatives to help people develop a more regular bowel pattern. Or the doctor may prescribe antidiarrheal medicines such as loperarnide or diphenoxylate to slow down the bowel and help control the problem. Bowel training helps some people re-learn how to control their bowels. In some cases, it involves strengthening muscles; in others, it means training the bowels to empty at a specific time of the day. Surgery may be an option for people whose fecal incontinence is caused by injury to the pelvic floor, anal canal, or anal sphincter. Various procedures can be done, from simple ones like repairing damaged areas, to complex ones like attaching an artificial anal sphincter or replacing anal muscle with muscle from the leg or forearm. People who have severe fecal incontinence that do not respond to other treatments may decide to have a colostomy, which involves removing a portion of the bowel. The remaining part is then either attached to the anus if it still works properly, or to a hole in the abdomen called a stoma, through which stool leaves the body and is collected in a pouch.

The artificial sphincter disclosed herein can be used to replace the patient's natural sphincters and when patient feels the need to have the bowel movement; patient has to activate the sphincter by simply applying the power to the sphincter actuator transcutaneously. The sphincter can be used alone or in combination with other conventional treatments for fecal incontinence.

Reflux Disorders

Gastroesophageal reflux disease, commonly known as GERD or acid reflux. It is a condition in which the liquid content of the stomach regurgitates (backs up or refluxes) into the esophagus. The liquid can inflame and damage the lining of the esophagus and cause esophageal inflammation and damage (esophagitis).

The body has ways (mechanisms) to protect itself from the harmful effects of reflux and acid. For example, most reflux occurs during the day when individuals are upright. In the upright position, the refluxed liquid is more likely to flow back down into the stomach due to the effect of gravity. In addition, while individuals are awake, they repeatedly swallow, whether or not there is reflux. Each swallow carries any refluxed liquid back into the stomach. The salivary glands in the mouth produce saliva, which contains bicarbonate. The bicarbonate neutralizes the acid that remains in the esophagus. However, at night while sleeping, gravity is not in effect, swallowing stops, and the secretion of saliva is reduced. Therefore, reflux that occurs at night is more likely to result in acid remaining in the esophagus longer and causing greater damage to the esophagus.

The major factors are the lower esophageal sphincter (LES), hiatal hernias (bulging of the esophagus between diaphragm and LES), esophageal contractions, and emptying of the stomach. The action of the lower esophageal sphincter (LES) is perhaps the most important factor (mechanism) for preventing reflux. Several different abnormalities of the LES have been found in patients with GERD. Two of them involve the function of the LES. The first is abnormally weak contraction of the LES, which reduces its ability to prevent reflux. The second is abnormal relaxations of the LES, called transient LES relaxations. They are abnormal in that they do not accompany swallows and they last for a long time, up to several minutes. These prolonged relaxations allow reflux to occur more easily. The transient LES relaxations occur in patients with GERD most commonly after meals when the stomach is distended with food. Transient LES relaxations also occur in individuals without GERD, but they are infrequent. The symptoms of uncomplicated GERD are primarily heartburn, regurgitation, and nausea. Some of the complications are ulcer, inflammation of the throat and larynx, and esophageal cancer.

Treatment for GERD includes life-style changes such as eating food at particular times of the day, not eating just before bed-time, eating food with less oil content, avoid eating fried food, less spicy food, etc. Drugs that are used include antacids, such as Turns; histamine antagonists such as cimetidine (Tagamet), ranitidine (Zantac), nizatidine (Axid), and famotidine (Pepcid); proton pump inhibitors (PPI) such as omeprazole (Prilosec), lansoprazole (Prevacid), rabeprazole (Aciphex), pantoprazole (Protonix), and esomeprazole (Nexium); pro-motility such as metoclopramide (Reglan); and foam barriers such as the combination of aluminum hydroxide gel, magnesium trisilicate, and alginate (Gaviscon).

Treatment option includes surgery. One of the procedure that is done to prevent reflux is technically known as fundoplication and is called reflux surgery or anti-reflux surgery. During fundoplication, any hiatal hernial sac is pulled below the diaphragm and stitched there. In addition, the opening in the diaphragm through which the esophagus passes is tightened around the esophagus. Finally, the upper part of the stomach next to the opening of the esophagus into the stomach is wrapped around the lower esophagus to make an artificial lower esophageal sphincter.

The artificial sphincter described herein can be used in combination with the conventional treatments for GERD, such as those listed herein. In preferred embodiments, the artificial sphincter is implanted above the LES around the esophagus and the induction coil is placed in the abdominal wall for powering the implant. The patient wears a power source and a transmitter coil similar to the one that is implanted in the abdominal wall. Microprocessor that is embedded in the coil senses the activities of swallowing, coughing, etc. and controls the sphincter opening and closing events as needed.

It is apparent to one skilled in the art that various changes and modifications can be made to this disclosure, and equivalents employed, without departing from the spirit and scope of the invention. Elements shown with any embodiment are exemplary for the specific embodiment and can be used on other embodiments within this disclosure.

What is claimed is:

1. An artificial sphincter comprising:
an electroactive polymer element and a support, said electroactive polymer element and support being configured to constrict a body cavity disposed therebetween.

2. The sphincter of claim 1 wherein said support comprises an encapsulating device comprising at least one passage for a body organ.

2. The sphincter of claim 2, wherein the passage is substantially circumscribed by a sheath.

3. The sphincter of claim 1, wherein the electroactive polymer element comprises an ion-exchange polymer metal composite.

4. The sphincter of claim 1, wherein the electroactive polymer element comprises a substantially flat surface.

5. The sphincter of claim 1, wherein the electroactive polymer element comprises a spring.

6. The sphincter of claim 1, further comprising a spring in mechanical communication with the electroactive polymer element.

7. The sphincter of claim 1, further comprising a power supply in electrical communication with the electroactive polymer element.

8. The sphincter of claim 1 wherein application of electrical current to said electroactive polymer element unconstricts said body cavity and removal of said electrical current constricts said body cavity.

9. The sphincter of claim 1 further comprising a coating to prevent tissue growth.

10. The sphincter of claim 1 further comprising a coating to promote tissue growth.

11. The sphincter of claim 1 further comprising an inductive coupling mechanism adapted to connect the electroactive polymer element to a power source.

12. An implantable control device comprising an electroactive polymer actuator, an enclosure, and a power management device; wherein said enclosure is configured to encompass a body cavity, said electroactive polymer actuator and said enclosure are configured to constrict said body cavity, and said power management device is adapted to connect to said electroactive polymer actuator.

13. The device of claim 12 wherein said electroactive polymer actuator is an ion exchange polymer metal composite material.

14. The device of claim 12 wherein said body cavity is a urethra, lower esophagus, lower gastro-intestinal tract, or rectum.

15. The implantable control device of claim 12 further comprising a coating to prevent tissue growth.

16. The sphincter of claim 12 further comprising a coating to promote tissue growth.

17. The implantable control device of claim 12 further comprising an inductive coupling mechanism adapted to connect the electroactive polymer actuator to the power management device.

18. A method of controlling passage of contents across a body cavity comprising:
implanting a control device around a body cavity, said device comprising an electroactive polymer actuator, an enclosure, and a power management device;
controlling a flow of contents in said body cavity, said control being performed by constricting and unconstricting said body cavity between said electroactive polymer actuator and said enclosure.

19. The method of claim 18 wherein said control of flow of contents in said body cavity is in response to transcutaneous feedback from said body cavity, said feedback being related to the contents in said body cavity.

20. The method of claim 18 wherein said body cavity is a urethra, lower esophagus, lower gastro-intestinal tract, or rectum.

21. The method of claim 18 wherein said control device is coated with an agent to prevent tissue growth around said control device.

22. The sphincter of claim 18 further comprising a coating to promote tissue growth.

23. The method of claim 18 wherein said control device is controlled by an inductive coupling mechanism.

24. A method of treating a disease using an artificial sphincter comprising:
implanting an artificial sphincter around a body cavity, said artificial sphincter comprising an electroactive polymer element and a support;
closing said body cavity with the artificial sphincter by applying a mechanical force on said body cavity between said support and said electroactive polymer element; and
opening said body cavity by transmitting an electrical signal to the electroactive polymer element.

25. The method of claim 24 wherein said disease is urinary incontinence, fecal incontinence, or reflux disease.

26. The method of claim 24 wherein said artificial sphincter is coated with an agent to prevent tissue growth around said artificial sphincter.

27. The sphincter of claim 24 further comprising a coating to promote tissue growth.

28. The method of claim 24 wherein said artificial sphincter is controlled by an inductive coupling mechanism.
